(19) United States
(12) Patent Application Publication
(10) Pub. No.: US 2013/0303907 A1
(43) Pub. Date: Nov. 14, 2013

(54) Device and System For Imaging and Blood Flow Velocity Measurement

(71) Applicant: Volcano Corporation, San Diego, CA (US)

(72) Inventor: Paul Douglas Corl, Palo Alto, CA (US)

(21) Appl. No.: 13/892,108

(22) Filed: May 10, 2013

Related U.S. Application Data
(60) Provisional application No. 61/646,080, filed on May 11, 2012.

(51) Int. Cl. A61B 8/06; A61B 8/00; A61B 8/12

(52) U.S. Cl. CPC A61B 8/06; A61B 8/12; A61B 8/4461; A61B 8/463
USPC 600/441; 600/443; 600/445

(57) ABSTRACT

Apparatuses, systems, and methods for intravascular ultrasound (IVUS) imaging and blood flow velocity measurement within a vessel using a rotational IVUS catheter are disclosed. The rotational IVUS catheter includes a transducer that is mounted to the catheter at an angle relative to the longitudinal axis of the catheter shaft, such that the imaging surface is substantially nonperpendicular to the angle of the blood flow. The IVUS imaging system includes the rotational IVUS catheter with the tilted transducer, sequencing hardware to generate a series of uniformly spaces transmit pulses and acquisitions per encoder pulse, and signal processing hardware to extract the phase from the ultrasound echo signals for velocity estimation at every pixel of the IVUS image. The system is configured to generate a hybrid IVUS image showing both structural and velocity characteristics of the vessel and the blood therein.

### CROSS-REFERENCE TO RELATED APPLICATION

This application claims priority to and the benefit of U.S. Provisional Application No. 61/646,080, filed May 11, 2012, which is hereby incorporated by reference in its entirety.

### BACKGROUND

The present invention relates generally to intravascular ultrasound imaging systems, and in particular to mechanically-scanned intravascular ultrasound (IVUS) imaging devices, systems, and methods directed to forming a cross-sectional image of a blood vessel and measuring the velocity of blood flow within the vessel.

Intravascular ultrasound imaging is widely used in interventional cardiology as a diagnostic tool for assessing a diseased vessel, such as an artery, within the human body to determine the need for treatment, to guide the intervention, and/or to assess its effectiveness. IVUS imaging uses ultrasound echoes to form a cross-sectional image of a vessel of interest. Typically, an ultrasound transducer on an IVUS catheter both emits ultrasound pulses and receives the reflected ultrasound echoes. The ultrasound waves pass easily through most tissues and blood, but they are partially reflected from discontinuities arising from tissue structures (such as the various layers of the vessel wall), red blood cells, and other features of interest. The IVUS imaging system, which is connected to the IVUS catheter by way of a patient interface module (PIM), processes the received ultrasound echoes to produce a cross-sectional image of the vessel where the transducer is located.

To establish the need for treatment, the IVUS system is used to measure the lumen diameter or cross-sectional area of the vessel. For this purpose, it is important to distinguish blood from vessel wall tissue so that the luminal border can be accurately identified. In an IVUS image, the blood echoes are distinguished from tissue echoes by slight differences in the strengths of the echoes (e.g., vessel wall echoes are generally stronger than blood echoes) and from subtle differences in the texture of the image (i.e., speckle) arising from structural differences between blood and vessel wall tissue. As IVUS imaging has evolved, there has been a steady migration towards higher ultrasound frequencies to improve the resolution in the display. But as ultrasound frequency is increased, there is diminished contrast between the blood echoes and vessel wall tissue echoes. At the 20 MHz center frequency used in early generations of IVUS, the blood echoes are very weak in comparison to the vessel wall echoes due to the small size of the red blood cell compared to the acoustic wavelength. However, at the 40 MHz ultrasound center frequency now commonly used for IVUS imaging, there is only a modest difference between blood and tissue echoes because the ultrasound wavelength at this higher frequency is closer to the dimensions of the red blood cells.

Another use of IVUS imaging in interventional cardiology is to help identify the most appropriate course of treatment. For example, IVUS imaging may be used to assist in recognizing the presence of a mural thrombus (i.e., coagulated blood attached to the vessel wall and stationary within the blood vessel) in an artery prior to initiating treatment. If a thrombus is identified in a region where disease has caused a localized narrowing of the arterial lumen, then the treatment plan could be modified to include aspiration (i.e., removal) of the thrombus prior to placing a stent in the artery to expand and stabilize the cross-sectional area of the vessel. In addition, the identification of a thrombus could lead the physician to order a more aggressive course of anti-coagulant drug therapy to prevent the subsequent occurrence of potentially deadly thrombosis. In a conventional IVUS image, however, there is very little difference in appearance between stationary thrombi and moving blood.

Another use of IVUS imaging in interventional cardiology is to visualize the proper deployment of a stent within an artery. A stent is an expandable cylinder that is generally deployed within the artery to enlarge and/or stabilize the lumen of the artery. The expansion of the stent typically stretches the vessel and displaces the plaque that otherwise forms a partial obstruction of the vessel lumen. The expanded stent forms a scaffold propping the vessel lumen open and preventing elastic recoil of the vessel wall after it has been stretched. In this context, it is important to recognize proper stent apposition; that is, the stent struts should be pressed firmly against the vessel wall. A poorly deployed stent may leave stent struts in the stream of the blood flow and these exposed stent struts are prone to initiate thrombus formation. Thrombus formation following stent deployment is referred to as “late stent thrombosis” and these thrombi can occlude the artery or break free from the stent strut to occlude a downstream branch of a coronary artery and trigger a heart attack.

In these examples of IVUS imaging, it is particularly useful to identify moving blood, and to distinguish the moving blood from the relatively stationary or static tissue or thrombi. Motion information can be helpful in delineating the interface between blood and vessel wall so that the luminal boundary can be more easily and accurately identified. Motion parameters such as velocity may be the most robust ultrasound-detectable parameters for distinguishing moving blood from stationary thrombi. In the case of stent malapposition, the observation of moving blood behind a stent strut is a clear indication that the stent strut is not firmly pressed against the vessel wall as it should be, possibly indicating a need to further expand the stent. In each of the aforementioned IVUS imaging examples, the addition of motion parameters to the traditional IVUS display of echo amplitude can improve the diagnosis and treatment of a patient.

Traditionally, IVUS catheters, whether rotational or solid-state catheters, are side-looking devices, wherein the ultrasound pulses are transmitted substantially perpendicular to the axis of the catheter to produce a cross-sectional image representing a slice through the blood vessel. The blood flow in the vessel is normally parallel to the axis of the catheter and perpendicular to the plane of the image. IVUS images are typically presented in a grey-scale format, with strong reflectors (vessel boundary, calcified tissue, metal stents, etc.) displayed as bright (white) pixels, with weaker echoes (blood and soft tissue) displayed as dark (grey or black) pixels. Thus, flowing blood and may appear very similar to soft tissue or static blood (i.e., thrombi) in a traditional IVUS display.

In non-invasive ultrasound imaging applications, Doppler ultrasound methods are often used to measure blood and tissue velocity, and the velocity information is used to distinguish moving blood echoes from stationary tissue echoes. Commonly, the velocity information is used to colorize the grey-scale ultrasound image in a format referred to as Doppler color flow ultrasound imaging, with fast moving blood tinted red or blue, depending on its direction of flow, and with stationary tissue displayed in grey scale.

Traditionally, IVUS imaging has not been amenable to Doppler color flow imaging since the direction of blood flow is predominantly perpendicular to the IVUS imaging plane. More specifically, Doppler color flow imaging and other Doppler techniques do not function well when the velocity of interest (i.e., blood flow velocity) is perpendicular to the imaging plane and perpendicular to the direction of ultrasound propagation, resulting in near zero Doppler shift attributable to blood flow. In the case of rotational IVUS, there is an added complication due to the continuous rotation of the transducer, which makes it problematic to collect the multiple echo signals from the same volume of tissue needed to make an accurate estimate of the velocity-induced Doppler shift. Various image correlation methods attempt to overcome the directional limitations of the Doppler method for intravascular motion detection, but are generally inferior to Doppler methods. Moreover, such image correlation techniques are not suitable for rotational IVUS because the rate of decorrelation due to the rotating ultrasound beam is comparable to the rate of decorrelation for the blood flow.

Accordingly, there is a need for apparatuses, systems, and methods that can produce intravascular ultrasound images using a mechanically-scanned ultrasound transducer, and that differentiate between moving blood and stationary tissue within a vessel. The apparatuses, systems, and methods disclosed herein overcome one or more of the deficiencies of the prior art.

### SUMMARY

Embodiments of the present disclosure describe a mechanically-scanned intravascular ultrasound (IVUS) imaging system that produces a rotational IVUS image with the addition of velocity data encoded as a color overlay on a grey-scale IVUS image to enhance the differentiation between moving blood echoes and stationary tissue echoes.

In one aspect, the present disclosure provides a rotational intravascular ultrasound system for imaging a vessel. The system comprises an ultrasound transducer rotationally disposed within an elongate member, and an actuator coupled to the transducer, the actuator moving the transducer through at least a portion of a revolution. The imaging system includes a control system controlling the emission of a sequence of ultrasound pulses and reception of the associated ultrasound echo signals. The control system processing the ultrasound echo signals to produce a cross-sectional image of the vessel based on both the echo amplitude and the Doppler frequency shift (indicative of the velocity of blood or other tissue within the vessel). In one embodiment, the actuator is coupled to the ultrasound transducer through a flexible drive cable extending substantially the entire length of the elongate member, the actuator continuously rotating the ultrasound transducer generally about a longitudinal axis of the elongate member.

In another aspect, the disclosure provides an ultrasound imaging system having a distal portion insertable into a vessel of a living body. The system comprising an elongate member having a longitudinal axis extending along a distal portion, the elongate member having an ultrasound transducer mounted adjacent to the distal portion such that an ultrasound pulse emitted by the transducer propagates away from the elongate member at a substantially non-perpendicular angle relative to the longitudinal axis. The ultrasound transducer being configured to receive ultrasound echo signals and convey these signals through a plurality of conductors extending between the ultrasound transducer disposed adjacent the distal portion to a connection assembly disposed adjacent an opposite proximal portion of the elongate member. The system further includes an actuator, which may be within the elongate member or external to the body, coupled to the ultrasound transducer. The actuator is configured to move the transducer through a range of positions extending over at least a portion of a revolution. In one embodiment the movement is continuous rotation about the longitudinal axis while in an alternative embodiment, the movement is an oscillatory action over a portion of a revolution. The system further includes a control system coupled to the connection assembly. The control system being configured to control the position of the ultrasound transducer and the timing of the ultrasound pulses emitted by the transducer. The control system receiving the ultrasound echo signals from the ultrasound transducer through the plurality of conductors and processing the echo signals to generate an image of the vessel. In one aspect, the vessel image includes amplitude data represented in grey-scale overlaid with colorized areas representative of the velocity data, derived by the control system from the Doppler frequency shifts detected in the ultrasound echo signals. In an alternative form, the grey-scale amplitude data is altered to reflect changes associated with the determination of the velocity data. In one form, pixels associated with velocities above a threshold value are suppressed or diminished in brightness to enhance the view of relatively stationary features of the blood vessel.

In another aspect, the present invention includes a method of imaging a vessel. The imaging method comprising positioning an elongate member having a distal portion with a longitudinal axis within the vessel, the catheter including an ultrasound transducer movably mounted within the distal portion. The method continues with emitting a sequence of ultrasound pulses from the transducer at a substantially non-perpendicular angle relative to the longitudinal axis while moving the transducer through at least a portion of a revolution with respect to the longitudinal axis. The method includes receiving the corresponding sequence of ultrasound echo signals from vessel features including blood within the vessel, processing the sequence of ultrasound echo signals to generate a single composite amplitude ray associated with a position along the arc, processing the sequence of ultrasound echoes to determine the velocity of vessel structures, and displaying a vessel image combining velocity and amplitude information. In one aspect, the display includes a color encoding of the velocity information combined with a grey-scale representation of the echo amplitude, while in an alternative aspect, pixels associated with velocities above a threshold value are suppressed or diminished in brightness in the displayed image. In a further aspect, the method can include automated determination of the vessel boundaries based on an algorithm utilizing the both velocity and amplitude information. In still a further aspect, the velocity information can be used to quantify blood flow within a vessel.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure. Throughout this description, like elements, in whatever embodiment described, refer to common elements wherever referred to and referenced by the same reference number. The characteristics, attributes, functions, interrelations ascribed to a particular element in one location apply to those elements when referred to by the same reference number in another location unless specifically stated otherwise.

The figures referenced below are drawn for ease of explanation of the basic teachings of the present disclosure only; the extensions of the figures with respect to number, position, relationship and dimensions of the parts to form the preferred embodiment will be explained or will be within the skill of the art after the following description has been read and understood. Further, the exact dimensions and dimensional proportions to conform to specific force, weight, strength, and similar requirements will likewise be within the skill of the art after the following description has been read and understood.

The following is a brief description of each figure used to describe the present invention, and thus, is being presented for illustrative purposes only and should not be limitative of the scope of the present invention.

FIG. 1 is a schematic illustration of a Doppler color flow rotational IVUS imaging system according to one embodiment of the present disclosure.

FIG. 2 is an illustration of a partially cross-sectional view of a rotational IVUS catheter according to one embodiment of the present disclosure.

FIG. 3 is an illustration of a partially cross-sectional view of the distal portion of the rotational IVUS catheter shown in FIG. 2 according to one embodiment of the present disclosure.

FIG. 4a is an illustration of a partially cross-sectional view of the rotational IVUS catheter shown in FIGS. 2 and 3 positioned within an artery according to one embodiment of the present disclosure.

FIG. 4b is an illustration of an IVUS grey-scale image according to one embodiment of the present disclosure.

FIG. 5a is an illustration of an IVUS velocity image according to one embodiment of the present disclosure.

FIG. 5b is an illustration of a hybrid color flow IVUS image according to one embodiment of the present disclosure.

FIG. 6 is a block diagram illustrating hardware components of the Doppler color flow rotational IVUS imaging system shown in FIG. 1 according to one embodiment of the present disclosure.

FIG. 7 is an illustration showing an ultrasound signal pattern of the Doppler color flow rotational IVUS imaging system shown in FIG. 1 according to one embodiment of the present disclosure.

FIG. 8 is a block diagram illustrating component parts of an echo processor of the IVUS imaging system shown in FIG. 1 according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure describes apparatuses, systems, and methods for producing Doppler color flow ultrasound images from a mechanically-scanned ultrasound transducer, deployable with a rotational intravascular ultrasound (IVUS) imaging system, to facilitate interpretation of cross-sectional images of a blood vessel of interest and to facilitate the qualitative or quantitative measurement of blood flow in the vessel. In particular, the disclosure describes one embodiment of the apparatuses, systems, and methods that produce a rotational IVUS image with the addition of velocity data encoded as a color overlay on a grey-scale IVUS image to enhance the differentiation between moving blood echoes and stationary tissue echoes.

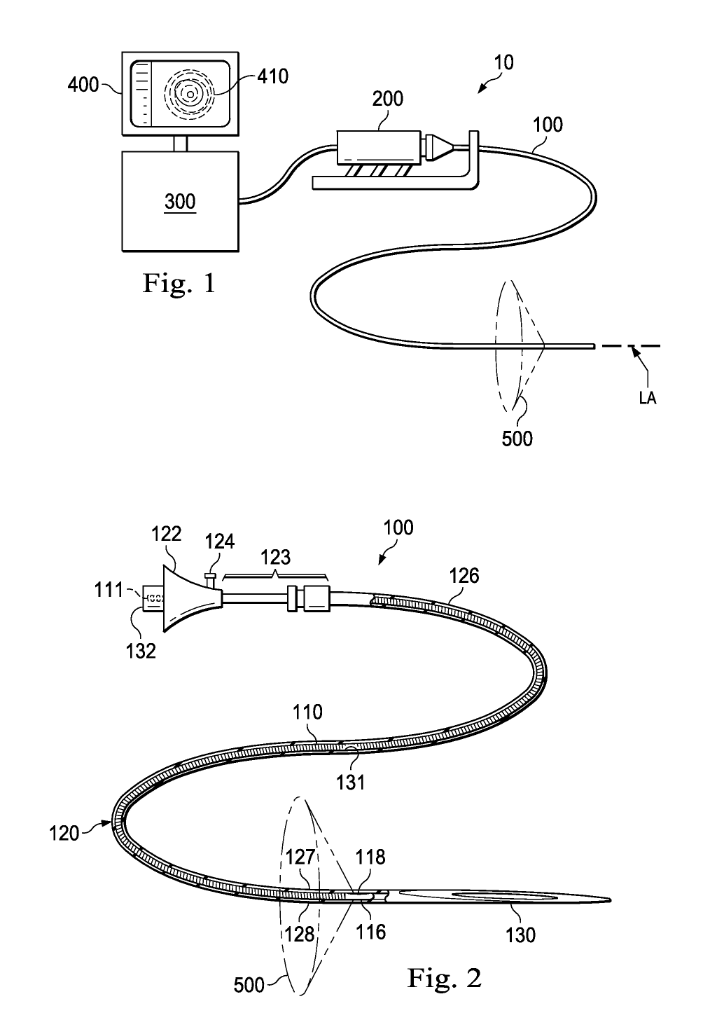

FIG. 1 illustrates a Doppler color flow rotational IVUS imaging system 10 according to one embodiment of the present disclosure. The main components of a rotational IVUS imaging system are the rotational IVUS catheter, the control system with its associated patient interface module (PIM), and a monitor to display the IVUS image. The key elements of the invention which distinguish it from a traditional rotational IVUS imaging system include a Doppler-enabled rotational IVUS catheter 100, a Doppler-capable IVUS imaging system 300 with associated patient interface module (PIM) 200, and a color monitor 400 to display the Doppler color flow IVUS image. In particular, the Doppler Color Flow Rotational IVUS Imaging System requires a modified rotational IVUS catheter 100 which includes an ultrasound transducer tilted at a modest angle away from a perpendicular to the axis of the catheter to provide a shallow conical imaging surface 500 instead of the traditional imaging plane which is nominally perpendicular to the axis of the catheter and the axis of the blood vessel.

The catheter 100 is an elongate member shaped and configured for insertion into a lumen of a blood vessel (not shown) such that a longitudinal axis LA of the catheter 100 substantially aligns with a longitudinal axis of the vessel at any given location along the length of the vessel. In that regard, the curved configuration illustrated in FIGS. 1 and 2 is for exemplary purposes only and in no way limits the manner in which the catheter 100 may curve in other embodiments. Generally, the catheter 100 is designed to be sufficiently flexible that it conforms to the natural curvature of the vessel into which it is inserted.

It will be understood that although the imaging system of FIG. 1 illustrates a catheter-based IVUS imaging system, the imaging components may be mounted on guidewires, treatment devices, implants, surgical tools, or other elongated members insertable into the body. It is understood that, in some instances, wires associated with the IVUS imaging system 10 extend from the control system 300 to the PIM 200 such that signals from the control system 300 can be communicated to the PIM 200 and vice-versa. In some instances, the control system 300 communicates wirelessly with the PIM 200. Similarly, it is understood that, in some instances, wires associated with the IVUS imaging system 10 extend from the control system 300 to the color monitor 400 such that signals from the control system 300 can be communicated to the color monitor 400 and/or vice-versa. In some instances, the control system 300 communicates wirelessly with the color monitor 400.

In a typical rotational IVUS catheter, a single ultrasound transducer element is mounted near the tip of a flexible driveshaft, which spins inside a plastic sheath inserted into the vessel of interest. The transducer element is oriented such that the ultrasound beam propagates generally perpendicular to the axis of the catheter. As the driveshaft rotates (typically at approximately 30 revolutions per second), the transducer is periodically excited with a high voltage pulse to emit a short burst of ultrasound. The same transducer then receives the returning ultrasound echoes reflected from various tissue structures, and the IVUS imaging system assembles a two dimensional display of the vessel cross-section from a sequence of several hundred of these ultrasound pulse/echo acquisition sequences occurring during a single revolution of the transducer. In a conventional IVUS imaging system, the imaging surface is nominally planar and generally perpendicular to the axis LA and the longitudinal axis of the blood vessel. An alternative configuration for a rotational IVUS catheter uses a rotating acoustic mirror combined with a stationary ultrasound transducer to produce a similar effect. In a still further configuration, an ultrasound transducer, mounted on a shaft extending from a motor or other actuator mounted in the distal portion of the catheter or guidewire may be used to mechanically scan the transducer through continuous rotation or over a portion of revolution. In response to electrical power or control signals, the motor or other actuator operates at predetermined speed to cause the shaft and transducer to rotate at a predetermined speed or oscillate at a predetermined rate. Examples of such systems are shown in U.S. Pat. Nos. 5,375,602 and 7,658,715, and in U.S. Patent Application Publication 2011/0071401, each of which is hereby incorporated by reference in its entirety.

The Doppler-enabled rotational IVUS catheter 100 includes an ultrasound transducer 118 tilted at a modest angle away from a perpendicular to the longitudinal axis LA, and it operates much as a conventional rotational IVUS catheter. As the driveshaft rotates (typically at approximately 30 revolutions per second), the transducer is periodically excited with a high voltage pulse to emit a short burst of ultrasound. During the brief time period following each transmitted pulse from the transducer 118, the echo signals from the surrounding tissue and blood are received by the transducer 118 and detected by the control system 300 by way of the PIM 200. The control system 300 then assembles a two-dimensional ultrasound image of a blood vessel cross-section from these hundreds of pulse/acquisition cycles occurring during a single revolution of the device. One cross-sectional image is produced for every rotation of the transducer 118, so the display is updated at approximately 30 frames per second to create the appearance of continuous real-time intravascular imaging. By virtue of the tilted transducer mount, the Doppler-enabled rotational IVUS catheter produces a shallow conical imaging surface 500 instead of the traditional imaging plane, and it may also acquire the Doppler frequency shift information needed to detect the blood velocity component parallel to the longitudinal axis of the catheter.

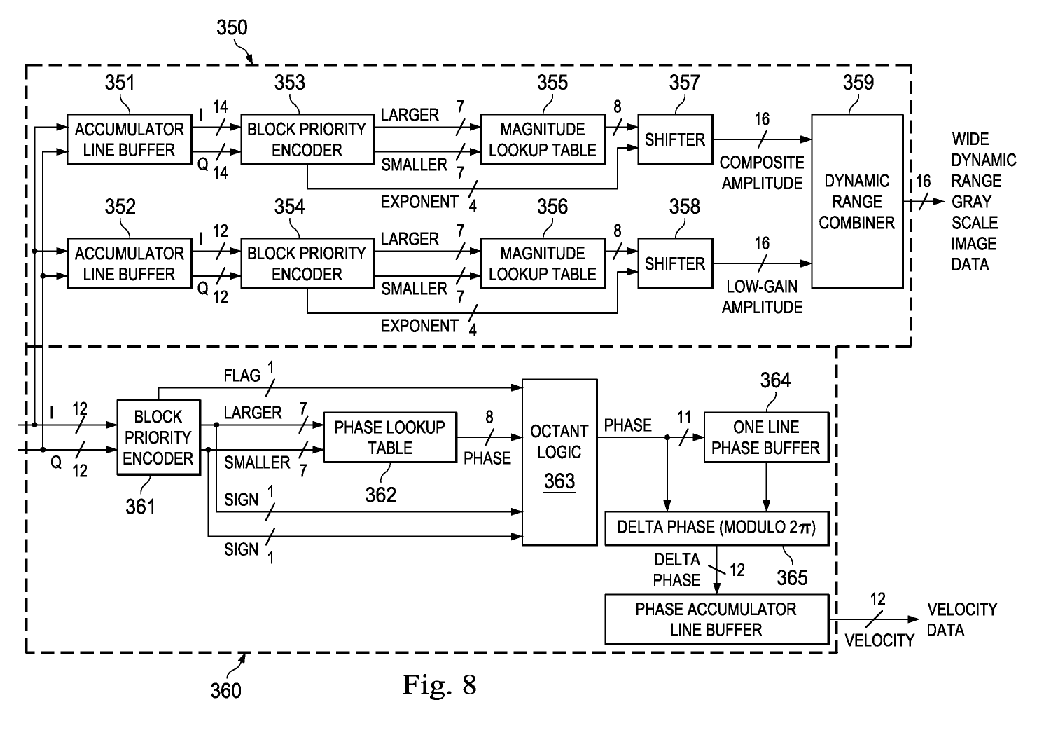

During operation of the imaging system 10, the control system 300 cooperates with the PIM 200 to generate the appropriate sequence of ultrasound transmit/receive cycles at each image angle to facilitate the extraction of velocity information from the sequence of echo signals. Specifically, as shown in FIG. 8, the Doppler-capable IVUS control system 300 includes signal processing hardware to simultaneously extract Doppler ultrasound velocity estimates to provide color encoding of the fast moving blood along with detecting the traditional echo amplitude data for producing the grey-scale IVUS display. The color monitor 400 displays a hybrid color flow image 410 comprised of the grey-scale IVUS image with the moving blood echoes highlighted in color to convey information regarding the magnitude and direction of blood velocity. The grey-scale IVUS image and/or the color flow image may be co-registered with other imaging data such as angiogram, MRI, and fluoroscopy as disclosed in U.S. Pat. No. 7,930,014, hereby incorporated by reference in its entirety.

FIG. 2 provides a more detailed view of the modified rotational IVUS catheter 100, which is optimized for Doppler color flow IVUS imaging. In some instances, the catheter 100 includes components or features similar to those of traditional rotational IVUS catheters, such as the Revolution® catheter available from Volcano Corporation and described in U.S. Pat. No. 8,104,479, or those disclosed in U.S. Pat. Nos. 5,243,988 and 5,546,948, each of which is hereby incorporated by reference in its entirety. In the illustrated embodiment, the catheter 100 includes a rotating imaging core 110 that is partially encased within a sheath 120. The rotating imaging core 110 terminates proximally in a rotational interface 111 that provides electrical and mechanical coupling to the PIM 200. The rotating imaging core 110 extends through the sheath 120 to distally terminate in a transducer housing 116, which houses the transducer 118. The sheath 120 includes a proximal bearing 122 coupled to a telescoping section 123, which is attached to a proximal portion 126 of the sheath 120. The proximal portion 126 is contiguous with a distal portion 127 of the sheath 120 that includes a window segment 128 and a tip assembly 130.

The proximal bearing 122 supports the rotational interface 111 of the rotating imaging core 110. In the illustrated embodiment, the proximal bearing 122 includes a port 124 for injecting saline into a lumen 131 of the sheath 120 and a fluid seal (not shown) to prevent the fluid from leaking out of a proximal end 132 of the sheath 120. The telescoping section 123 permits the sheath 120 to be reduced or extended in length, causing the rotating imaging core 110 to be correspondingly advanced or retracted with respect to the window segment 128 of the sheath. This telescoping configuration facilitates longitudinal pullback of the transducer 118 through a segment of vessel that is to be examined by the rotational IVUS imaging system 10.

The proximal 126 and distal 127 portions of the sheath partially or fully encase the rotating imaging core 110. The proximal portion 126 comprises a robust, flexible, cylindrical tube that extends from the telescoping section 123 to the window segment 128. The window segment 128 is structurally contiguous with the proximal shaft 126, but it is formed of different material than the proximal portion 126 of the sheath. The window segment 128 may be composed of a material (or combination of materials) that has an acoustic impedance and a sound speed particularly well-suited for conducting the ultrasound beam from the transducer 118 out into the blood vessel with minimal reflection, attenuation, or beam distortion. The tip assembly 130 extends distally from the window segment 128 and is shaped and configured to engage with a conventional coronary guidewire to enable the IVUS catheter 100 to be easily directed into a vessel of interest and to be easily removed from the guidewire.

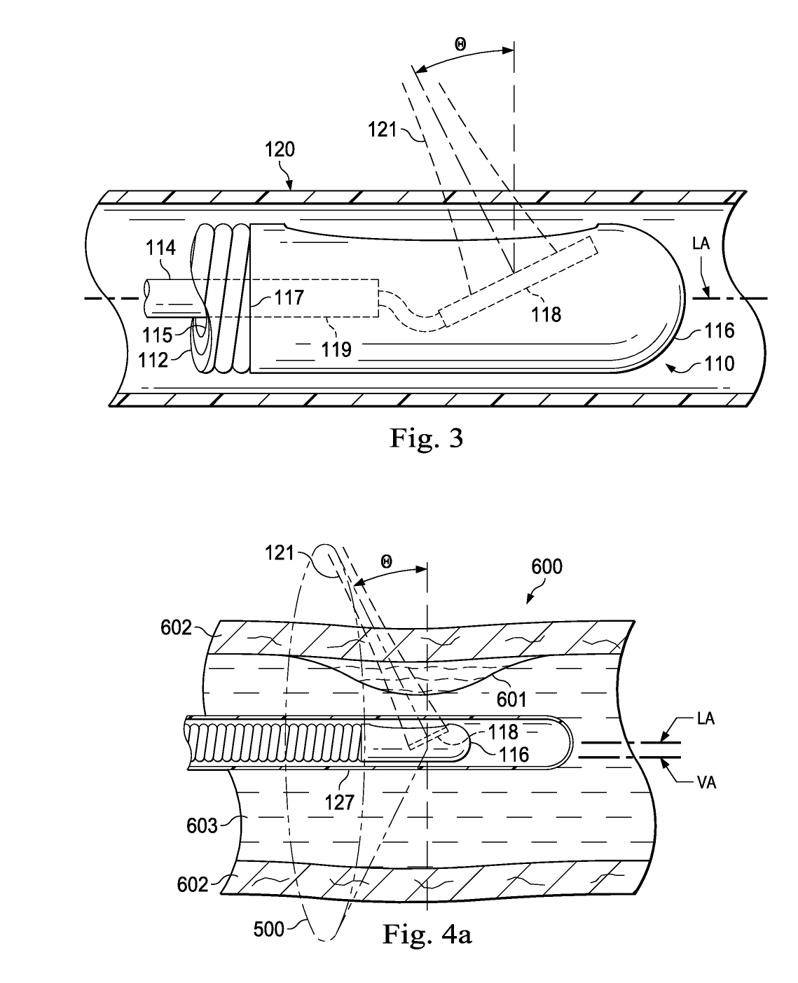

FIG. 3 provides a more detailed view of a distal end of the rotating imaging core 110. In the illustrated embodiment, the rotating imaging core 110 includes a flexible driveshaft 112 composed of two or more layers of counter wound stainless steel wires, an electrical cable 114 threaded through an inner lumen 115 of the flexible driveshaft 112, a transducer housing 116 coupled to a distal end 117 of the flexible driveshaft 112, and an ultrasound transducer 118 mounted inside the transducer housing 116. Although not pictured in FIG. 3, the driveshaft 112 extends the length of the rotating imaging core 110 to the rotational interface 111 (shown in FIG. 2). In alternative embodiments, the driveshaft 112 may be composed of a different material. The rotational interface 111 includes an electrical connector (not shown) and a mechanical structure (not shown) to connect a proximal end of the imaging core 110 to a rotating PIM drive assembly (not shown). A proximal end (not shown) of the electrical cable 114 is attached to the electrical connector portion of the rotational interface 111, and a distal end 119 of the electrical cable 114 passes through the inner lumen 115 of the flexible driveshaft 112 to connect to the ultrasound transducer 118 located inside the transducer housing 116.

The ultrasound transducer in a conventional rotational IVUS catheter is mounted substantially in line with the catheter axis LA so that the ultrasound beam emerges substantially perpendicular to the axis of the catheter. In practice, the transducer is frequently mounted at a slight angle in order to reduce the strength of the echo from the catheter sheath. The echo received by the transducer from the catheter sheath) will be strongest when the reflector is parallel to the transducer face and the echoes from different portions of the reflector arrive back at the transducer in phase with one another. If the transducer surface is tilted at an angle such that there is at least one wavelength of path length difference across the axial length of the transducer, then the echoes from the different portions of the sheath will tend to cancel and the echo will be reduced.

As an example of the degree of transducer tilt preferred for a conventional rotational IVUS catheter, the window or aperture width for a typical rotational IVUS catheter is approximately twelve wavelengths (for example, a 500 μm transducer length and approximately 40 μm wavelength at a 40 MHz transducer center frequency). To introduce one wavelength of round trip path length difference across the aperture would require one-half wavelength of tilt over the same width, or an angle of approximately 1/24 radians (approximately 2.5°). With optimum sheath design, the sheath reflection can be small enough that no transducer tilt is needed. Transducer tilt angles in the range of 0° to 8° are common for conventional rotational IVUS catheters.

As described above, in a conventional rotational IVUS catheter, the ultrasound beam emerges from the transducer substantially perpendicular to the longitudinal axis of the catheter, and as the imaging core rotates, the ultrasound beam sweeps out a substantially planar imaging surface to produce an ultrasound image of a cross-sectional slice through the vessel. In this traditional configuration, there is minimal Doppler frequency shift introduced by the moving blood, since the blood motion is substantially parallel to the axis of the vessel, parallel to the axis of the catheter, and accordingly perpendicular to the direction of propagation of the ultrasound beam. Since there is minimal Doppler frequency shift attributable to blood motion, it is difficult to derive a blood flow velocity estimate by Doppler methods using a conventional rotational IVUS catheter.

However, tilting the transducer mounting angle away from the traditional orientation causes a significant component of the blood velocity to be aligned with the ultrasound propagation direction such that the blood velocity (generally parallel to the catheter longitudinal axis LA) can be detected by measuring a Doppler frequency shift. For example, in FIG. 3, the mounting angle of the transducer 118 for the Doppler enabled rotational IVUS catheter 100 is tilted prominently from the longitudinal axis LA of the rotating imaging core 110, such that the ultrasound beam 121 emerges from the catheter at a transducer tilt angle θ of 10° to 30° with respect to a perpendicular to the catheter axis LA, and more preferably at an angle of 15° to 25°. In one embodiment, the transducer tilt is set to an angle of 20°. FIG. 3 shows the transducer 118 tilted toward the proximal shaft 126, but the tilt could be in the opposite direction as well, toward the tip assembly 130. In alternate embodiments, the catheter 100 may be configured to have any of a variety of transducer tilt angles depending upon the particular application.

The tilted transducer orientation required to produce a significant Doppler frequency shift in the ultrasound echoes from the moving blood transforms the traditional imaging plane into a shallow conical imaging surface. But with a modest transducer tilt, only slight geometric distortion is introduced in projecting this conical imaging surface onto a planar display, and this distortion does not significantly impair the physician's ability to interpret the image. There are two competing considerations for choosing the transducer tilt angle θ for the catheter 100: (1) the larger the tilt angle, the greater (and more readily detectable) will be the Doppler component in the ultrasound echo, and (2) the larger the tilt angle, the greater will be the geometric distortion when a conical imaging surface is projected onto a planar display.

The Doppler shift measured by an ultrasound system is proportional to the cosine of the angle between the direction of the motion and the direction of propagation of the ultrasound beam. In the idealized circumstance, where the longitudinal axis LA of the catheter is aligned with the longitudinal axis of the vessel, and where the velocity of blood flow is parallel to the longitudinal axis of the vessel as well, the angle between the direction of the blood flow and the direction of the ultrasound beam is the complement of the transducer tilt angle θ. Accordingly, the Doppler shift is proportional to the sine of the transducer tilt angle θ. For a zero tilt angle, there is no significant Doppler shift, and the velocity information cannot be obtained from traditional Doppler signal processing. The theoretical maximum Doppler shift would be obtained with a transducer tilt angle of 90°, but that would preclude the possibility for IVUS imaging since the ultrasound beam would then be aligned with the axis of rotation (axis LA). At a modest tilt angle of 30°, the Doppler shift is 50% of the theoretical maximum, and a reasonable IVUS image from the shallow conical imaging surface 500 (shown in FIGS. 1 and 2) can still be obtained.

For the intracoronary IVUS application, the Doppler velocity data is important for its role in helping to differentiate blood from tissue, hence the importance of distinguishing the Doppler shift of fast moving blood from the Doppler shift of slow moving tissue. In color flow imaging applications throughout most of the body (e.g., liver, carotid, or peripheral artery), the tissue motion is negligible, so the velocity threshold for classification of an echo as a moving blood echo can be very low. However in the case of coronary imaging, the tissue motion can be quite prominent, and it is more difficult to reliably distinguish tissue motion from blood flow. In general, it is helpful for distinguishing the Doppler shift of fast moving blood from the Doppler shift of slow moving tissue to use a larger transducer tilt angle to increase the Doppler shift for the blood with its predominantly axial velocity, while having little effect on the Doppler shift of the tissue with its predominantly radial velocity.

Although the motion of the heart muscle is quite rapid during early systole when the ventricles contract, the IVUS catheter tends to move with the heart by virtue of its capture within the coronary artery. Thus, the relative motion between the catheter and the surrounding tissue is usually significantly less than the absolute motion of the heart. An example of a fast movement of the IVUS catheter with respect to the heart would be for the catheter to shift one vessel diameter (˜3 mm) during the approximately 100 msec that constitutes the early portion of systole. The corresponding relative tissue velocity in this case would be ˜3 cm/sec. Throughout most of the cardiac cycle, and in the majority of locations throughout the epicardial arterial tree, the actual tissue velocity will be much less than this estimate. In particular, in the coronary arteries, blood flow is most significant (typically in the range of 10 cm/sec to 100 cm/sec) during diastole, the portion of the cardiac cycle when the heart motion is at its minimum (as the heart muscle gradually relaxes). Accordingly, in some embodiments, it is desirable to gate the Doppler color flow imaging with the ECG to capture blood flow measurements only during diastole, when the blood flow is maximum, and the heart motion (and relative tissue velocities) are at a minimum.

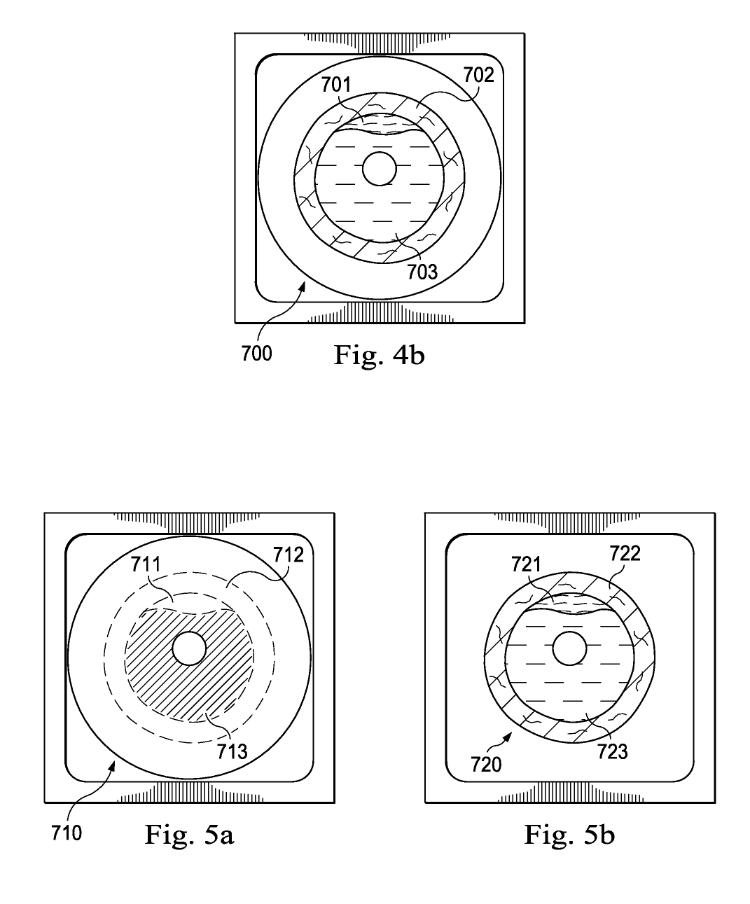

FIG. 4a illustrates the distal portion 127 of the Doppler-enabled rotational IVUS catheter 100 positioned within a vessel 600, which includes a lesion 601 attached to the vessel wall 601 within a lumen 602. The catheter 100 includes the transducer 118 mounted at a significant tilt angle within the housing 116. In FIG. 4a, the catheter 100 is shown positioned within the moving blood 603 of the vessel 600 such that the axis LA of the catheter 100 is substantially parallel to a longitudinal axis VA of the vessel 600 (and to the direction of the blood flow). In the pictured embodiment, the ultrasound beam 121 emerges from the transducer 118 at a tilt angle θ with respect to a perpendicular from the longitudinal axis VA of the vessel, and as the rotating imaging core 110 rotates, the ultrasound beam 121 sweeps out the conical imaging surface 500 to produce a cross-sectional ultrasound image of the vessel.

The choice of the transducer tilt angle for Doppler color flow rotational IVUS imaging should consider the robustness of the Doppler velocity measurement in the face of misalignment between the catheter axis and the axis of the blood vessel, as well as the ability to distinguish the Doppler shift of fast moving blood from the Doppler shift of slow moving tissue. In the course of normal clinical use, there may be a misalignment between the axis LA of the catheter 100 and the axis VA of the vessel (and the direction of blood flow). If that misalignment is comparable to the transducer tilt angle θ, then the Doppler shift in a portion of the vessel might be reduced to zero where the catheter misalignment cancels the transducer tilt angle θ. However, if the transducer tilt angle θ is significantly greater than the typical range for catheter misalignment, then the system 10 will retain a robust capability for estimating blood velocity across the entire vessel lumen. Because the human anatomy may include significant tortuosity in regions where IVUS imaging is commonly used (e.g., but not by way of limitation, the coronary arteries), it is difficult to predict the largest misalignment that can exist between the vessel axis VA and the catheter axis LA. In one example, a large misalignment that might be found in clinical practice would be the equivalent of a 1 millimeter (mm) diameter catheter traversing a 3 mm in diameter vessel lumen over a 10 mm length of vessel, corresponding to a likely maximum misalignment angle of approximately 12°. Over much of the epicardial arterial tree, however, the actual misalignment angle would be less than this maximum likely value. Nevertheless, it would be helpful for maintaining a robust Doppler signal if the transducer tilt angle θ was greater than 12°. Based on this consideration, the transducer tilt angle θ should preferably be greater than 15° to allow a small margin above the 12° maximum likely misalignment angle predicted above. More preferably, the transducer tilt angle θ should be approximately 20° to provide a greater margin of tolerance for catheter to vessel misalignment.

As illustrated in FIG. 4a, the ultrasound beam 121 emerges from the transducer 118 at a significant angle with respect to the longitudinal axis VA of the vessel 600, and as the transducer 118 rotates, the ultrasound beam 121 sweeps out a conical imaging surface 500 to produce an ultrasound image 700 of the vessel 600, as shown in FIG. 4b. It is important to note that the imaging surface 500 is not perpendicular to the direction of the blood flow along the longitudinal axis VA. As the rotating imaging core 110 and the transducer 118 rotate inside the sheath 120, the transducer 118 sends the ultrasound beam 121 toward the vessel wall 602. Ultrasound echoes from tissue elements or structures within the vessel 600, including the lesion 601, the vessel wall 602, and the moving blood 603, are received by the transducer 118. These ultrasound echoes are transmitted to the control system 300 via the PIM 200 (shown in FIG. 1), and the IVUS system 10 processes the echoes to create a tomographic grey-scale image 700 (cross-sectional slice) of the vessel, including representations of the lesion 701, the vessel wall 702, and the blood 703.

In most respects, the grey-scale image 700 is substantially the same as that produced by the traditional IVUS method using a non-tilted or marginally tilted transducer, except for a slight geometric distortion arising from projecting the conical imaging surface onto a planar display. Since the ultrasound image produced from the conical surface 500 is typically displayed on a planar video monitor, there is a geometric distortion introduced in the conical to planar transformation. The degree of distortion can be quantified by a figure of merit which represents the discrepancy between radial and tangential distance measurements on the distorted planar display. The distortion figure of merit can be calculated as one minus the cosine of the tilt angle. A zero tilt angle produces a planar imaging surface with no distortion, while a tilt angle of 20° produces 6% distortion. A modest degree of distortion will not interfere with the qualitative interpretation of the image which requires the identification of the inner and outer borders of the vessel wall structures and assessment of the general character of the echoes from lesions within the vessel wall. Any quantitative measurements, such as lumen diameter or plaque cross-sectional area to be made from the distorted planar display can be easily corrected by applying the appropriate geometric formula to remove the conical distortion from the calculation. For the preferred range of tilt angles θ from 10° to 30°, the visual distortion ranges from 1.5% to 13%, while for the more preferred range of tilt angles θ from 15° to 25°, the visual distortion ranges from 3% to 9%.

Therefore, the choice of the transducer tilt angle θ for the Doppler Color Flow Rotational IVUS imaging system 10 may involve consideration of the following factors: (1) the robustness of the Doppler velocity measurement in the face of misalignment between the catheter axis LA and the longitudinal axis of the blood vessel, (2) the ability to differentiate Doppler shift of fast moving blood from the Doppler shift of slow moving tissue, and (3) the degree of distortion of the IVUS image when the conical image surface is projected onto a planar display (with a view to minimize such distortion). A compromise tilt angle can be chosen wherein the image distortion due to projection of the conical imaging surface onto the planar display is acceptably small, while the Doppler shift is large enough that it provides a robust blood velocity measurement, tolerant of small misalignments between the catheter axis LA and vessel axis VA, and sufficient to differentiate fast moving blood from stationary or slow moving tissue.

In the grey-scale image 700 depicted in FIG. 4b, the appearance of the blood 703 is slightly different from the appearance of the vessel wall 702 or the lesion 701, but the distinction between blood echoes and vessel wall echoes is not great. Particularly at the higher ultrasound frequencies preferred for high resolution IVUS imaging, the distinction between blood echoes and vessel wall or plaque echoes is subtle. The strength of an ultrasound echo is strongly influenced by the size of the reflecting object compared to the ultrasound wavelength. For example, at the 20 MHz ultrasound frequency used by some of the older rotational IVUS imaging systems, the echo from the vessel wall tissue is typically much stronger than the echo from the moving blood, since the blood cells are much smaller (approximately 6 μm in diameter) than the coherent tissue structures that make up the vessel wall (e.g., collagen fibers, smooth muscle cells, tissue layers, etc.) and much smaller than the ultrasound wavelength (approximately 75 μm). In contrast, at the 40 MHz ultrasound frequency commonly used for rotational IVUS imaging today, the contrast between vessel wall or plaque tissue echoes and blood echoes is diminished since the shorter acoustic wavelength (approximately 40 μm) more closely approaches the diameter of the blood cells. The low image contrast between the blood 703 and the vessel wall tissue 702 or plaque 701 may make it difficult to identify the boundaries of the lumen and to quantify anatomic parameters such as diameter or cross-sectional area of the vessel 600, which are helpful in guiding the treatment of the coronary artery disease. Note that the black-on-white depiction of the tomographic image depicted in FIG. 4b is the negative of the white-on-black image typically shown on the IVUS display monitor.

It is important to note that noninvasive color flow imaging systems cannot take advantage of high ultrasound frequencies, such as 40 MHz, due to the frequency-dependent attenuation of the ultrasound in tissue which severely limits the penetration depth. Furthermore, noninvasive color flow imaging systems cannot take advantage of high ultrasound frequencies, such as 40 MHz, since the high ultrasound frequency results in large Doppler frequency shifts that necessitate a high pulse repetition frequency and short period between successive ultrasound pulses, once again limiting the usable penetration depth. However, for rotational IVUS imaging, the shallow penetration depth (approximately 5 mm) permits the use of a high pulse repetition frequency adequate to capture the maximum velocity likely to be encountered in a physiological environment. For rotational IVUS imaging, the required penetration depth is only about 5 mm, and the attenuation in blood, even at a 40 MHz ultrasound frequency, is low enough to allow adequate signal to noise ratio for such a shallow penetration depth.

To improve the diagnostic value of the IVUS image 700, the Doppler enabled IVUS catheter 100 and the Doppler-capable IVUS control system 300 utilize a separate signal processing path operating in parallel to the standard imaging path to provide velocity information for the various components within the vessel 600. While the standard image processing algorithm translates the amplitude of the echo signal into grey-scale brightness on the display image 700, a parallel signal processing path extracts a velocity estimate for every pixel of the display image 700 from the information contained in the Doppler frequency shifts of the echo signals.

FIG. 5a depicts the image that would be obtained if the imaging system 10 was programmed to display an image of the velocity estimates extracted from the ultrasound echoes received by the transducer 118 instead an image of the amplitudes of the ultrasound echoes received by the transducer 118. The lesion representation 711 and the vessel wall representation 712 of the velocity image 710 would indicate low velocities for the relatively static lesion 601 and vessel wall tissue 602, respectively, while the relatively fast-moving blood 603 within the vessel lumen 602 would be prominently highlighted by the blood velocity representation 713.

In practice, the separate grey-scale IVUS image 700 and velocity image 710 may be difficult to interpret, but a synergistic image may be produced by combining the echo amplitude and velocity information together in a hybrid Doppler color flow image 720, as shown in FIG. 5b, in which the echo amplitude is encoded as image brightness and velocity is encoded in color. For example, but not by way of limitation, the echo velocities may by displayed in the hybrid color flow image 720 in shades of red and blue for antegrade and retrograde flow, respectively, while relatively stationary or slow-moving tissues may be displayed in shades of grey. In the hybrid color flow image 720, the stationary lesion 721 and vessel wall 722 appear in grey-scale much the same as in a conventional IVUS image, while the representation 723 of moving blood is highlighted in red by virtue of its velocity-related Doppler frequency shift. The enhanced image contrast between the blood 723 and the vessel wall 722 in the color flow image 720 makes it much easier (compared to traditional IVUS imaging) for the user and/or the system 300 to identify the boundary of the vessel lumen 602 and to quantify anatomic parameters such as diameter or cross-sectional area of the vessel 600, which are important for guiding the treatment of the coronary artery disease.

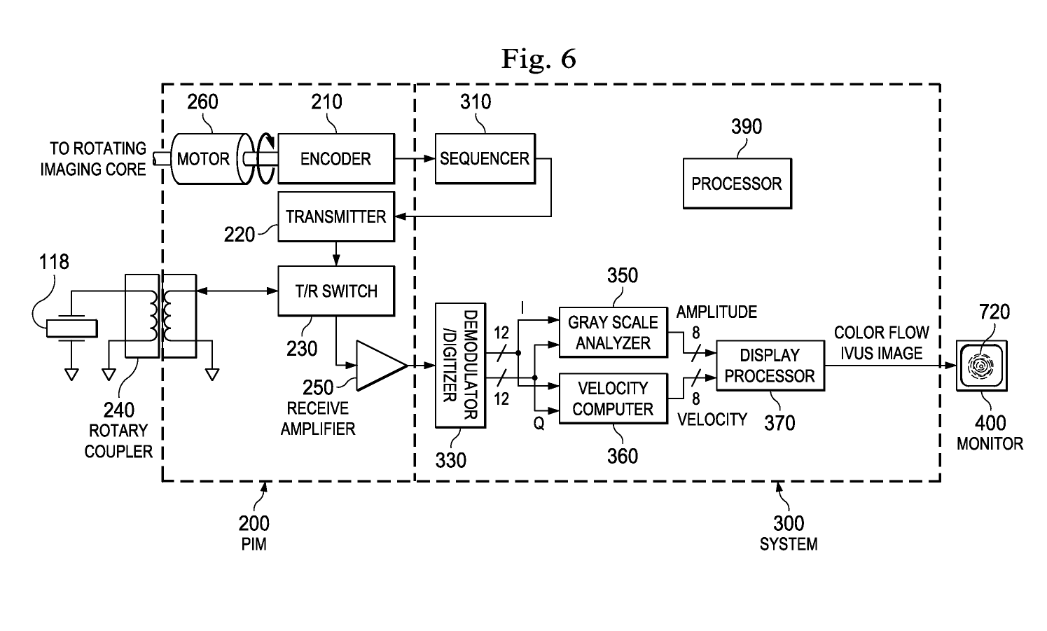

FIG. 6 presents a block diagram of the individual hardware components of the Doppler color flow rotational IVUS imaging system 10 according to one embodiment of the present disclosure. In the illustrated embodiment, the PIM 200 includes an encoder 210, a transmitter 220, an ultrasound transmit/receive (T/R) switch 230, a rotary coupler 240, an amplifier 250, and a motor 260. The control system 300 includes a sequencer 310, a demodulator/digitizer 330, a grey-scale analyzer 350, a velocity computer 360, a display processor 370, and a processor 390, which coordinates and controls the operation of the IVUS imaging system.

The encoder 210, which is coupled to the motor 260 that drives the rotating imaging core 110 (not shown), generates pulses at regular intervals throughout the rotation of the imaging core 110 (i.e., typically 512 pulses per revolution). Instead of each encoder pulse generating a single trigger pulse as in a traditional IVUS system, each encoder pulse triggers a sequence of multiple transmit triggers (such as, by way of not-limiting example, 2 to 16 triggers) via the sequencer 310. Each transmit trigger initiates a pulse from the transmitter 220 which passes through the ultrasound T/R switch 230 to reach the ultrasound transducer 118 by way of the rotary transformer 240. The T/R switch 230 protects the delicate circuitry of the amplifier 250 from the high-voltage transmit pulses while permitting the low amplitude echo signals to pass freely to the amplifier input. The rotary transformer 240 allows the transmit pulses and the echo signals to pass between the stationary elements of the PIM 200 and the rotating imaging core 110 that carries the transducer 118.

For rotational IVUS imaging at 30 frames per second, the orientation of the transducer 118 is constantly changing, making it difficult to collect the repeated measurements from a single direction that are preferred for creating a Doppler velocity estimate. However, given the high speed of sound propagation through tissue compared to the scanning rate, together with the short penetration depth for IVUS imaging of approximately 5 mm, there is sufficient time to include a sequence of several ultrasound transmit/receive cycles for each imaging angle within the IVUS display. The duration of this rapid sequence of pulses can be sufficiently short that several successive transmit/receive cycles will capture echoes from substantially the same tissue/blood volume such that the Doppler frequency shift can be extracted from the echoes received during this sequence of transmit/receive cycles.

For a period of time after each transmit pulse (e.g., typically approximately 10 μsec), the amplifier 250 receives a low level echo from the transducer 118 and applies the appropriate time dependent gain to produce an amplified echo signal. The amplitude of the echo signal versus time (relative to the transmit pulse) is representative of the reflectivity of the (reflecting) tissue as a function of distance from the transducer 118. In addition, information regarding the motion of the tissue is encoded in the small changes, particularly the phase shift, between one echo signal and the next within a sequence. The amplifier 250 transmits the amplified echo signals to signal processing hardware in the control system 300 for processing.

For Doppler processing, it is convenient to transform the radio frequency (RF) echo waveform into a baseband representation according to methods well-known to those skilled in the art, wherein the transducer center frequency is shifted down to DC, and the echo signal is represented as digitized samples of a complex modulation waveform comprised of in-phase (I) and quadrature (Q) components. The demodulator/digitizer 330 transforms the amplified echo signal from the amplifier 250 into a baseband representation of the signal comprising digitized samples of the I and Q components of the complex modulation waveform. This function can be performed in the analog domain using a pair of mixers, followed by a pair of analog-to-digital converters to provide digital samples of the I and Q components. Alternatively, the demodulation step can be performed in the digital domain by direct sampling of the RF echo waveform with a high speed analog-to-digital converter, followed by digital filtering to produce digital samples of the I and Q components of the complex modulation waveform.

The grey-scale analyzer 350 and the velocity computer 360 process the multiple echo signals from a single sequence as a group, and use the information contained in the sequence of echo signals to (1) detect the echo amplitude as a function of depth to generate a single ray or radial line of the grey-scale image (commonly referred to as an A-line), and (2) to calculate the Doppler-derived velocity for each position along that ray, respectively. Specifically, the grey-scale analyzer 350 uses the information contained in the sequence of echo signals to detect the echo amplitude as a function of depth to generate a single A-line of the grey-scale image with a low noise level and wide dynamic range, while the velocity computer 360 calculates the Doppler-derived velocity estimate for each position along that A-line from the small phase changes from one echo signal to the next within a single sequence. In theory, the velocity data could be used to produce a velocity image 710 of a cross-section through the vessel 600, but in practice, it is convenient to combine the echo amplitude data with the velocity data to produce a hybrid color flow image 720 combining the grey-scale IVUS image with velocity information encoded as shades of red and blue (for antegrade and retrograde flow), and with stationary and slow moving tissues displayed in shades of grey. As the rotating imaging core 110 and transducer 118 rotate within the sheath 120, the IVUS imaging system 10 builds a complete cross-sectional image 700 (commonly referred to as a B-scan image) of the artery 600 from the succession of A-lines from the grey-scale analyzer 250. The amplitude and velocity data are also combined into color-coded A-lines and scan converted in the display processor 370 for display as the hybrid color flow image 720 on the color monitor 400.

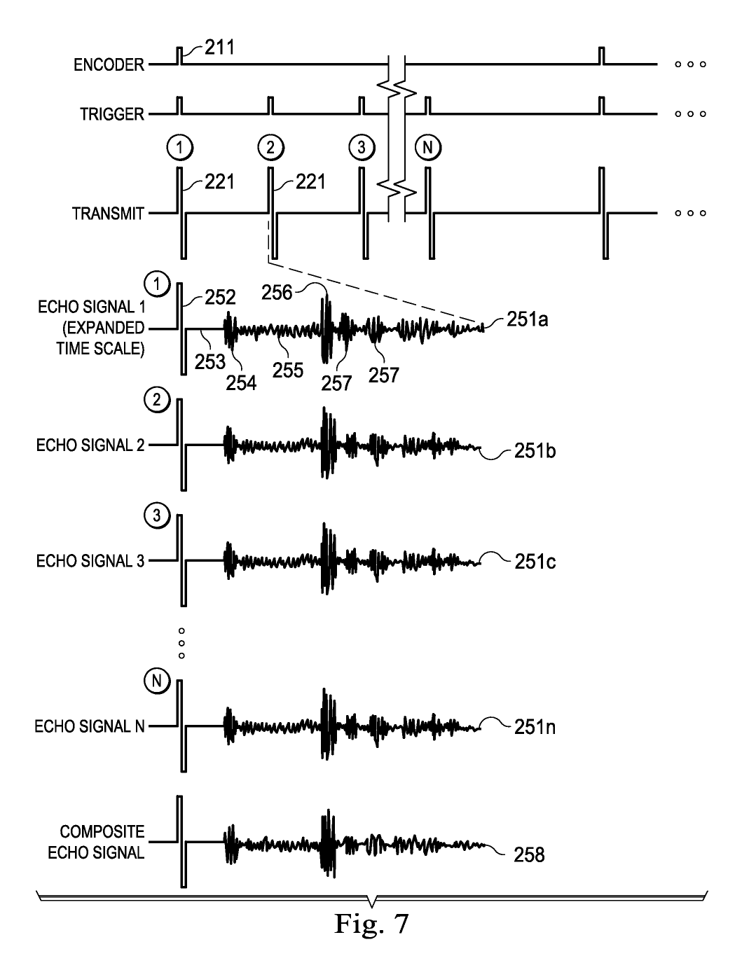

FIG. 7 illustrates the nature of the typical signals produced by the Doppler color flow rotational IVUS imaging system 10 (shown in FIG. 1) according to one embodiment of the present disclosure. In order to capture the Doppler velocity information, the imaging system 10 ideally triggers a sequence of N evenly spaced transmit pulses 221 and acquisition sequences (instead of the single transmit pulse and acquisition per encoder pulse used in the conventional IVUS imaging system). Therefore, each encoder pulse 211 triggers a sequence of typically 2 to 16 high-voltage transmit pulses 221 that are uniformly spaced in time. Since the transmit pulses within a sequence are relatively closely spaced in time, the corresponding ultrasound beams cover substantially the same tissue, and the phase change at any given point can be largely attributed to motion. In some instances, the number of pulses would likely range from 2 to 16, with 4 pulses providing a good compromise for producing robust velocity estimation without substantially limiting the penetration depth. In some instances, it may be helpful to precede the pulse/acquisition sequence with a dummy transmit pulse (having no acquisition) so that the first acquisition will have a similar level of residue from the previous transmit pulse compared to subsequent acquisitions. The IVUS amplitude data (grey-scale data) may be derived from an average of the multiple acquisitions, or from just a single acquisition. In some instances, it may be desirable to make the velocity acquisitions relatively limited in depth because the vessel lumen has a limited size, and then allow the amplitude acquisitions to be much longer to capture the deep tissue echoes.

Following each of the high voltage transmit pulses 221 in the sequence, the amplifier 250 (not shown in FIG. 7) receives a low level echo from the transducer 118 (not shown in FIG. 7) and applies appropriate time dependent gain to produce one of the first amplified echo signal 251a, second 251b, third 251c, and continuing through the Nth amplified echo signal 251ₙ. Each of these signals 251 exhibits similar features, including a high amplitude artifact 252 from the transmit pulse 221, a brief quiet period 253 as the ultrasound propagates through the saline within the sheath, and a strong sheath echo 254 from the sheath 120 (not shown in FIG. 7). After the sheath echo 254, there is a period 255 of weak blood echoes from the blood within the vessel, a strong wall echo 256 from the inside of the vessel wall, and moderate tissue echoes 257 from the vessel wall tissue. Each echo signal 251 has the general character of an amplitude modulated waveform with a carrier frequency corresponding to the center frequency of the ultrasound transducer 118. The modulation amplitude of the signal 251 versus time corresponds to the echogenicity of the tissue as a function of depth.

Each sequence of transmit trigger pulses 221 initiates the acquisition of a sequence of echo signals 251, and although the echo amplitude can be easily derived from a single echo signal from within that sequence, it is more difficult to extract the velocity information from just one echo signal. However, the velocity can be estimated by analyzing how the echo signal changes from one echo signal to the next within a sequence. For stationary or slow-moving tissue, the echo signal 251 changes very little from one echo signal 251 to the next within a sequence, since the pulses within a sequence are so closely spaced in time that there is little tissue motion over that short interval. Furthermore, the rotation of the transducer 118 over this short interval is small enough compared to the dimensions of the ultrasound beam 121 (shown in FIG. 4a) that the beam 121 covers substantially the same volume of tissue for each of the transmit/receive acquisitions within a single sequence. However, for fast-moving tissue (e.g., flowing blood), there is enough movement over the course of a sequence of pulses that a phase-sensitive detector within the control system 300 can extract a velocity estimate from the small phase changes between one echo signal 251 (e.g., 251ₙ) and the next (e.g., 251ₙ₊₁) within a sequence.

It is advantageous under circumstances described below, to arrange the sequence of acquisitions such that the first amplified echo signal 251a in the sequence is acquired with a lower analog gain setting in the amplifier 250 compared to the gain used for the second 251b through the Nth 251ₙ acquisitions. In this case, the first acquisition, acquired with a low analog gain setting, accurately capture the echoes from strong reflectors such as calcified plaque or metal stent struts without the distortion that arises when the amplifier is driven into saturation by a strong echo signal acquired with a high amplifier gain setting. The subsequent acquisitions within the sequence are collected using the higher gain setting to faithfully capture the low amplitude echo signals from blood, soft plaques, and other low reflectivity tissues. The first acquisition, captured using a low analog gain setting, is not particularly useful for Doppler velocity estimation since the weak echoes from blood and soft tissue are likely to be partially obscured by the quantization noise from the analog-to-digital converter.

However, this low-gain acquisition is useful for generating wide dynamic range grey-scale image data, and it also serves to initialize the reverberations within the transducer, the catheter, and the medium, thereby reducing the Doppler artifact that can arise from such reverberations or the absence thereof. Reverberations arise from acoustic signals originating from the transmit pulse(s) prior to the most recent one. For grey-scale IVUS imaging, these reverberations are generally of little consequence since they are greatly diminished over the time between acquisitions and only generate a small perturbation in the amplitude of the echo signal. However, these small perturbations can give rise to phase artifacts that may be significant for low level echoes that are of great interest with respect to blood flow, causing artifacts in the blood velocity estimates. Excluding the first echo acquisition from the Doppler velocity computation ensures that each of the subsequent acquisitions used for the velocity algorithm includes a similar pattern of reverberation that will tend to introduce zero Doppler shift, at least to the extent that the reverberation is consistent from pulse to pulse. In this case, only the transmit pulse from the first acquisition affects the reverberations in subsequent acquisitions, and the amplifier gain for the first acquisition is irrelevant to the reverberations. Acquiring one low-gain acquisition as the first in a sequence facilitates wide dynamic range grey-scale IVUS imaging without compromising the velocity measurement potential of the system.

Although the amplitude signal can be derived from just a single echo signal 251 chosen from the sequence of echo signals acquired, it is advantageous to construct a composite echo signal 258 by signal averaging or a similar technique applied to the entire sequence of echo waveforms to provide an improved signal-to-noise ratio. The envelope derived from this composite echo signal 258 exhibits improved signal-to-noise ratio compared to that derived from just a single echo waveform 251. Signal processing across the ensemble of echo waveforms within a sequence is facilitated by processing the data in the digital domain where the multiple waveforms can be readily stored, retrieved, and processed.

The amplitude and velocity information can be independently presented as separate images of echo amplitude and Doppler velocity over the field of view, but it is preferred to combine these two sets of information into the hybrid color flow image 720 (shown in FIG. 5b) combining the grey-scale IVUS image with the velocity data encoded as shades of red and blue (for antegrade and retrograde flow, respectively), with stationary and slow-moving tissues displayed in shades of grey. Furthermore, the combined amplitude and velocity data can be further analyzed to extract anatomic features of the vessel 600 (shown in FIG. 4a) such as the lumen border or functional measures such as volumetric flow. Such added analyses, facilitated by the availability of the combined echo amplitude and Doppler velocity data, further enhance the value of the Doppler color flow rotational IVUS imaging system 10.

In particular, the combined amplitude and velocity data may be utilized, for example but without limitation, by the imaging system 10 to enhance suppression of blood echoes, luminal border and cross-sectional area detection, quantitative blood flow measurements, and thrombus detection. The imaging system may enhance the contrast between the blood echoes and the vessel wall by using color to highlight the moving blood or by simply deemphasizing the moving blood by reducing the brightness of the fast-moving blood elements of the image. For example, to suppress blood echoes from the final image 720, the imaging system 10 diminishes the brightness of the echoes that contain a significant velocity component so that the vessel lumen 602 (shown in FIG. 4a) appears empty or darker than normal (as represented by the moving blood representation 723 in the image 720), thereby enhancing the distinction between the luminal blood 723 and the vessel wall 722 or plaque 721.

To enhance luminal border detection, the imaging system 10 uses the velocity data to improve the algorithm for automatic (computer-based) detection of the lumen border and the lumen cross-sectional area. These can be determined by manually tracing the lumen borders or by placing markers at intervals around the vessel border, but it is highly advantageous if those measurements are automatically provided by a computer algorithm that identifies the border on its own. Some IVUS imaging systems include such automated measurement algorithms, but these frequently require human intervention to improve the quality of the border detection. Such a system is described in U.S. Pat. No. 6,945,938 hereby incorporated by referenced herein in its entirety. Providing velocity information to the automatic border detection algorithm can improve the quality of the result and reduce the need for tedious manual tracing of the borders.

As described above, in the traditional IVUS imaging system, the differentiation between moving blood and stationary thrombus may be very subtle. There may a slight difference in the temporal appearance of the speckle pattern, but there is often very little difference in the echogenicity of blood versus thrombus (particularly fresh thrombus). However, velocity provides a very strong signature to differentiate moving blood from stationary thrombus, and Doppler color flow imaging by the imaging system 10 utilizing blood velocity data may greatly improve the detection of thrombus.

To estimate quantitative blood flow, the imaging system 10 numerically integrates the blood velocity data over the cross-sectional area of the vessel lumen 602 to provide a quantitative measurement of volumetric blood flow within the artery 600 (shown in FIG. 4a). The combination of IVUS imaging with Doppler velocity measurement makes it possible to accurately quantify blood flow. Blood flow calculation provides functional parameters to supplement the anatomic measurements provided by the IVUS hybrid image 720. By comparing the blood flow under hyperemic and resting conditions, for example, the coronary flow reserve can be computed as the ratio of the two flows to provide an important figure of merit for cardiac performance. The use of a pharmacologic agent, such as, by way of non-limiting example, adenosine, to stimulate maximum hyperemia in the vessel 600 may facilitate the calculation of coronary flow reserve, an important diagnostic value.

In addition, the imaging system 10 may extend the dynamic range of the grey-scale IVUS image by using the same pulse sequence used to provide the information needed for measuring Doppler frequency shift. In a healthy artery, a clear IVUS image may be obtained with relatively modest dynamic range. However, in the pathological conditions of greatest interest to the physician, a wider dynamic range is frequently needed. In diseased arteries, there are often deposits of calcium within a plaque, and these calcium deposits produce strong echoes that may tend to dominate the image and obscure the nearby low level echoes. Similarly, IVUS is frequently used for imaging arteries where metal stents have been placed, and the metal stent struts produce strong echoes which tend to obscure the vessel wall image behind the struts. The wide dynamic range feature offers a significant benefit under these pathological conditions by reducing the noise level in the image, enhancing the visibility of weak reflectors, and reducing the image artifacts arising from strong reflectors such as calcium deposits or stent struts. Extending the dynamic range of the IVUS signals can make it easier to detect the weak echoes from soft tissue while simultaneously detecting the strong echoes from metal stent struts or calcified plaques embedded in the vessel wall. Extending the dynamic range of the IVUS signals is discussed in more detail below with respect to FIG. 8.

FIG. 8 shows a detailed view of the signal processing algorithm implemented in the circuitry of the grey-scale analyzer 350 and the velocity computer 360 (shown in FIG. 6) according to one embodiment of the present disclosure. The grey-scale analyzer includes the amplitude detection circuitry to derive the grey-scale image information, while the velocity computer includes the phase detection circuitry used to derive the velocity information for color coding the hybrid Doppler color flow image. The input to both the grey-scale analyzer and velocity computer is a sequence of amplified echo signals as illustrated in FIG. 7, converted to a baseband I/Q representation for signal processing convenience, and represented as 12-bit binary values (11-bits plus sign) in this example. The embodiment detailed in FIG. 8 is well-suited to implementation in a field programmable gate array (FPGA), which can incorporate the entire digital signal processing chain for the Doppler color flow rotational IVUS imaging system 10 in a single integrated circuit device.

The amplitude detection circuitry could be as simple as calculating the envelope of a single A-line at a time and forwarding that envelope data on to the display processor. But because the phase detection circuitry used for the Doppler velocity computation preferably uses a sequence of echo signal acquisitions covering substantially the same volume of tissue, it is advantageous to use the same multiple acquisitions to improve the signal-to-noise ratio and dynamic range available for the grey-scale display. IVUS imaging produces a wide dynamic range of echoes, ranging from the weak echoes from blood or soft tissue to the strong echoes from calcified plaque or metal stent struts. One method to expand the dynamic range available for display is to increase the signal-to-noise ratio by averaging multiple echo signals together to reinforce the coherent echoes from the tissue compared to the incoherent noise present in the individual echo signals. Another method for expanding the dynamic range is to acquire echo signals with different analog gain settings, and to then splice together the low gain samples from strong reflectors with high gain samples from weak reflectors, including digital compensation for the different analog gains. Additional noise reduction may be achieved by applying a nonlinear algorithm across the sequence of echo signal acquisitions to reject isolated impulsive noise spikes.

In the embodiment shown in FIG. 8, the amplitude detection circuitry incorporates an accumulator/line buffer 351 which averages together a sequence of echo signals in a baseband I/Q format to produce a composite echo signal, which is also in baseband I/Q format, having improved signal-to-noise ratio and dynamic range compared to a single echo signal. The signal-to-noise ratio is generally improved as the square root of the number of signals averaged together, and, in this example, averaging up to 16 signals within a single sequence would require an increase from 12-bits for the original exemplary echo signal resolution up to 14-bits for the composite echo signal with improved signal-to-noise ratio.

A separate low gain line buffer 352 stores an echo signal, which is also in baseband I/Q format, acquired using a lower amplifier gain compared to the gain used for acquiring the other echo signals in the acquisition sequence. The echo signal acquired with lower amplifier gain captures a low distortion representation of strong echoes from calcified plaques or stent struts that might saturate the amplifiers and acquisition circuitry when a higher amplifier gain is used for the other echo signal acquisitions in the sequence. In a typical example, the low gain amplifier setting would be −12 dB relative to the high gain setting (a factor of one-quarter), with correspondingly reduced distortion from amplifier or signal acquisition stage saturation due to strong echoes. If the signal amplitude is less than one-quarter of the maximum low gain value, then the low-noise composite echo signal derived from the multiple high gain acquisitions provides the best representation of the echo signal, but for any echo amplitude above that threshold, the composite echo signal is likely to be saturated, and the low gain acquisition samples should be used instead.

The amplitude of each of these buffered signals (the composite echo signal and the low gain echo signal) can be calculated using a variety of methods known to those skilled in the art, but the method described herein is well-suited for implementation in an FPGA, since it requires relatively simple logic and small lookup tables stored in the FPGA memory, and it operates directly on echo signal waveforms captured in baseband I/Q format. Essentially identical circuitry is shown for envelope detection of the accumulated composite echo signal and the buffered low gain echo signal, but this circuitry could be shared by time-multiplexing the signals from those two separate signal paths through one set of envelope detection circuitry in order to reduce the required FPGA resources.

The first step of the envelope detection process is to convert the linear representation of the baseband I and Q values into a more compact representation (requiring fewer bits) in order to simplify the subsequent calculations and reduce the size of the required lookup tables. A block priority encoder 353 or 354 converts an I/Q sample pair into a floating point format, using a shared exponent for both samples. The block priority encoder determines which of the I and Q samples is the largest, preserving the most significant bits of that value as the mantissa and using simple logic to calculate the associated exponent (power of 2) required for the floating point representation of the original sample. The smaller of the two samples is shifted by the number of bit positions specified by the shared exponent, and the high order bits become the mantissa for the smaller of the two samples. In this illustrative example, the 12- or 14-bit I and Q samples (11 or 13 bits plus sign) are converted to floating point representations, each with a sign (not needed for amplitude calculation) plus 7-bit mantissa, and with a shared 4-bit exponent.

At this point, the benefit of the block priority encoder becomes apparent in the small size of the magnitude lookup table (LUT) 355 or 356 required for calculating the magnitude of the I/Q sample pair as the square-root of the sum of the squares of the two values. In this illustrative example, two 7-bit mantissas necessitate a modest-sized 8-kbyte (2¹³×1 byte) LUT to provide the square-root of the sum of the squares of the two mantissas. Only 13 bits are required to address the magnitude LUT because the most significant bit of the larger mantissa is omitted since it is always a 1 and the sign bits are ignored since they don't affect the magnitude calculation. Without this block floating point approach or another efficient method, an impractically large 128-megabyte (2²⁶×2-byte) LUT would be needed to calculate the square-root of the sum of squares of a pair of 14-bit composite I and Q samples (sign bits are ignored for the magnitude calculation). Once the mantissa of the magnitude is provided by the magnitude LUT 355 or 356, the exponent (representing a common factor shared by both I and Q values) is applied through the shifter 357 or 358 to reverse the floating point conversion and restore a linear representation of the magnitude of the envelope for the corresponding signal (either the composite echo signal or the low gain echo signal).

Finally, to provide wide dynamic range grey-scale image information to the display processor 370, the dynamic range combiner 359 splices together the low-amplitude, low-noise envelope data from the composite echo signal with the high-amplitude, low-distortion envelope data from the low-gain echo signal. The result is exceptionally wide dynamic range for the grey-scale image data, facilitating the display of weak tissue and blood echoes visible above a very low noise floor, while strong echoes from stent struts or calcified plaques appear without saturation. The dynamic range combiner may be as simple as a comparator that switches between either of the two signal sources based on the strength of the echo at that particular point. If the signal is weak, then the low-noise composite echo signal is used as the source for the grey-scale information, and if the signal is strong enough to approach the full-scale maximum for the composite echo signal, the dynamic range combiner switches seamlessly to the low-distortion echo signal acquired using reduced amplifier gain.

More advanced schemes for combining these two echo signals may include a transition zone between the two signal sources, wherein low amplitude echoes are derived solely from the low-noise composite echo signal, strong echoes are derived from the low-distortion, low-gain echo signal, and over a broad intermediate range, the grey-scale information is provided by interpolation between the two signal sources using amplitude-dependent coefficients. For example, if the low-gain sample is less than one-sixteenth of the full-scale amplitude, then the low-noise composite sample should be well below its full scale limit of one-quarter of the low-gain maximum, and the composite sample with its low noise level is used alone. For samples greater than one-quarter of the full-scale amplitude of the low-gain acquisition, the composite sample is likely to be beyond its full scale maximum value and the low-gain sample may be used alone to provide a low-distortion sample of the high amplitude echo amplitude. For the samples in the transition range between one-sixteenth and one-quarter of the full-scale amplitude of the low-gain acquisition, a weighted average of the composite and low-gain samples may be used, with amplitude-dependent coefficients gradually phasing in one or the other source based on the echo amplitude at a particular point. At the output of the dynamic range combiner, the amplitude signal covers a very wide dynamic range with reduced noise compared to the noise level expected from a single acquisition. The wide dynamic range A-line is encoded as a 16-bit integer capable of encoding a dynamic range on the order of 96 dB.

In the embodiment shown in FIG. 8, the velocity computer 360 provides the velocity information used to color code the hybrid Doppler color flow image. The velocity computer utilizes the same input data stream as the grey-scale computer, that is a sequence of amplified echo signals as illustrated in FIG. 7, converted to a baseband I/Q representation for convenience, and represented as 12-bit binary values (sign+11-bits) in this example. The phase detection circuitry used for estimating the Doppler velocity can be implemented using a variety of algorithms known to those skilled in the art. The algorithm shown in FIG. 8 relies on simple logic together with modestly sized lookup tables to perform the nonlinear function required to calculate the phase, and it is well-suited to implementation in a field programmable gate array (FPGA), which can implement the entire digital signal processing chain for a Doppler color flow rotational IVUS imaging system in a single integrated circuit device.

A variety of methods are known to those skilled in the art for extracting a Doppler-derived velocity estimate from a sequence of echo signals. These methods have been applied extensively to noninvasive Doppler color flow ultrasound imaging systems, but heretofore this technology had not been thought to be applicable to rotational IVUS imaging systems for the reasons discussed above. The phase detection circuitry is designed to extract a Doppler velocity estimate for each radial position along an A-line of the image, from the sequence of echo signal acquisitions that is obtained from that angular position. The movement of blood or tissue at a particular depth along a ray of the image is encoded in the sequence of echo signals as a rate of change in phase of the echo signal at that radial position. The phase detection circuitry is designed to determine the phase change at every sample depth for each echo signal acquisition (ignoring the low-gain acquisition if that feature is implemented), to calculate the change in phase from one echo signal to the next within a sequence, to accumulate the change in phase over the series of echo signal acquisitions within the sequence, and to estimate the tissue or blood flow velocity from the rate of change in phase according to the Doppler equation.

The first step of the phase detection process is to convert the linear representation of the baseband I and Q values into a more compact representation (requiring fewer bits) in order to simplify the subsequent calculations and reduce the size of the required lookup tables. A block priority encoder 361 converts an I/Q sample pair into a floating point format, using a shared exponent for both samples. The block priority encoder determines which of the I and Q samples is the largest, and it preserves the most significant bits of that value as the larger mantissa while generating an I>Q comparison flag according to which of the two values is larger. The priority encoder uses simple logic to calculate the associated exponent (power of 2) required for the floating point representation of the larger sample, and the smaller of the two samples is shifted the number of bit positions specified by the shared exponent, with the high order bits then becoming the mantissa for that smaller of the two samples. In this illustrative example, the 12-bit I and Q samples (11-bits plus sign) are converted to floating point representations, each with a sign plus 7-bit mantissa, and with a shared 4-bit exponent (which is not needed for phase detection).

At this point, the benefit of the block priority encoder becomes apparent in the small size of the phase LUT 362 required for calculating the phase of the I/Q sample pair as the arctangent of Q/I. In this illustrative example, two 7-bit mantissas necessitate a modest-sized 8-kbyte (2\hat{0}×1 byte) LUT to provide the arctangent computation over one octant. Only 13 bits are required to address the magnitude LUT because the most significant bit of the larger mantissa is omitted as it is always 1, and the exponent is ignored since it is common to both I and Q and does not affect the ratio of Q/I. The one-octant phase angle from the phase LUT is expanded to a full four-quadrant phase angle by octant logic 363 by utilizing the two sign bits to identify one of the four quadrants and utilizing the I>Q comparison bit from the block priority encoder to identify which of the I/Q sample pair is larger. Without this block floating point approach or other efficient method, an impractically large 16-megabyte (2²⁴×1-byte) LUT would be needed to compute the arctangent of Q/I for a pair of 12-bit I and Q samples.

The Doppler velocity is estimated for each radial position along an A-line by measuring the rate of change of phase at that point. This may be accomplished by buffering the phase from one line of acquisition in the one-line phase buffer 364, and then subtracting this buffered phase data, sample by sample, from the next line of phase data as it is loaded into the one-line phase buffer replacing the prior line of buffered phase data. The subtraction operation used to calculate the phase change is performed (modulo 2π) in the delta phase block 365. The phase change as calculated covers a range of 2π, but this phase change must be properly interpreted to distinguish positive velocity (antegrade flow) from negative velocity (retrograde flow). If there is no a priori knowledge regarding the direction of flow, then an unbiased approach would be to interpret the phase change values to represent a range from −π to +π. If there is some a priori knowledge that flow is strictly one direction, then a biased approach may be to assign all phase changes to be either positive or negative, according to the assumptions about the directional nature of the blood flow. There can be an intermediate interpretation as well, for example if the velocity is predominantly in one direction, the phase change could be interpreted to represent a range from −π/2 to +3π/2.

The range of velocities that can be unambiguously interpreted is limited by the requirement that the Doppler frequency shift must not exceed one-half of the pulse repetition frequency between successive transmit pulses within a sequence (for the unbiased case described previously). In a typical example, the transmit pulses within a sequence are spaced 10 μsec apart, corresponding to a 100 kHz pulse repetition frequency, and the corresponding maximum Doppler frequency is 50 kHz. The Doppler equation can be used to translate this maximum Doppler frequency into a maximum detectable blood velocity. The Doppler equation states:

$$velocity = \frac{c}{2 cos cos α} \frac{( f {Doppler}_{}}{{f}_{Center}} )$$

In this equation, c represents the speed of sound in blood, 1540 m/sec, α is the angle between the direction of blood flow and the direction of ultrasound propagation, that is, the complement of the transducer tilt angle (for example, α would be 70° for a typical transducer tilt angle of 20° anticipated for the Doppler-enabled rotational IVUS catheter). For rotational IVUS catheters, the typical ultrasound center frequency is 40 MHz, and for the maximum Doppler frequency of 50 kHz, the calculated maximum measurable blood velocity is ±2.80 m/sec. This range of velocities covers most clinical conditions where the device is likely to be used, with a blood flow velocity generally less than 1.0 m/sec in a relatively unobstructed coronary artery under resting conditions. This range of velocities may be extended by implementing a biased delta phase detector as described previously.

The delta phase block 365 calculates the difference in phase between corresponding samples from two successive acquisitions of phase data, and it applies the selected interpretation of phase over a 2π range. The cumulative phase change is then calculated over a sequence of acquisitions to provide a robust estimate of the rate of change of phase at each point along the corresponding A-line. This rate of phase change can be interpreted as a velocity estimate by applying a constant factor derived from the Doppler equation according to methods known to those skilled in the art.

After a complete acquisition sequence is processed, the output of the velocity computer 360 is a single line of velocity data corresponding to the single A-line of grey-scale amplitude data provided by the grey-scale analyzer 350. As subsequent acquisition sequences are processed, additional lines of grey-scale and velocity data are produced and these lines of data are used to paint a complete tomographic image of the vessel, including color encoded velocity information to aid in the interpretation of the image.

Referring back to FIG. 6, the display processor 370 performs a variety of functions, including grey-scale mapping (e.g., log compression, gamma correction, etc.) to transform the wide dynamic range amplitude data into display brightness in a format that is pleasing to the eye and easy to interpret, scan conversion to transform the polar scanning format of the rotational IVUS catheter into a raster format for compatibility with a conventional monitor, and combination of the grey-scale and velocity data into a hybrid color flow image format. There are a number of schemes known to those skilled in the art for combining the grey-scale IVUS data with the corresponding velocity information to produce a Doppler color flow image. One simple scheme is to establish a threshold for the maximum likely tissue velocity, and then to assume that any velocity greater than this threshold must represent moving blood.

In some embodiments, a negative threshold and a positive threshold may be used, wherein any velocity below the negative threshold is assumed to retrograde flow, any velocity above the positive threshold is assume to be antegrade flow, and any velocity between the positive and negative thresholds is assumed to be stationary or slow-moving tissue. This velocity threshold scheme can be used to generate a simple, three level color mask, with blue tint applied to the grey-scale value for any velocity below the negative threshold, red tint for any velocity above the positive threshold, and no tint (grey) for any velocity values between these threshold values representing stationary or slow-moving tissue. In other embodiments, the color flow imaging may use the mask to define the vessel boundaries and support border detection, virtual histology, or a de-speckling algorithm to more clearly distinguish the blood from the stationary tissue.

In some embodiments, a more elaborate scheme may be used with shades of red through yellow encoding positive velocities, with shades of blue through green encoding a range of negative velocities, and with stationary and slow moving tissue receiving a neutral (grey) tint.

Another option might be to forego the color display entirely, and simply use the velocity information to identify moving blood, and then to suppress the grey-scale brightness of the blood speckle to more clearly differentiate the moving blood from the stationary or slow-moving vessel wall. Advanced algorithms might even integrate the velocity map over the cross-section of the artery to provide a quantitative measurement of volumetric flow in the artery.

In some embodiments, the velocity threshold may be chosen to separate moving blood with axial velocities in the 10 to 200 centimeters per second (cm/sec) range from moving tissue with typical velocities on the order of 3 cm/sec or less. The Doppler component will be only 30% of the axial velocity due to the transducer tilt angle, while vessel wall motion is likely to be in the direction of the ultrasound beam where it will cause a maximum clutter signal.

It is important to note that while the apparent tissue motion due to rotation of the transducer in a rotational IVUS catheter creates an obstacle to use of image correlation methods for motion detection, this apparent velocity does not produce a significant Doppler frequency shift because the apparent motion is in a tangential direction, perpendicular to the direction of propagation of the ultrasound beam. Therefore, the transducer rotation does not present a fundamental impediment to Doppler-based velocity measurement.

For application in coronary IVUS imaging, the Doppler velocity data is important for its role in helping to differentiate blood from tissue. The anatomy and physiology of the coronary arteries creates unique blood flow characteristics. In the coronary arteries, blood flow occurs predominantly during the diastolic phase of the cardiac cycle, during which tissue motion is at a minimum because the heart muscle is relaxed. In some instances, early diastole is a preferred phase of the cardiac cycle in which to use the blood velocity to assist with border detection. During early diastole, the velocity information provides maximum differentiation between the stationary tissue and the moving blood, since blood velocity is at its maximum and the heart motion is minimal.

Diastole is also a preferred time for measuring the artery cross-sectional area and diameter, while the distortion of the lumen due to compression of the heart muscle is minimized. In general, it is advantageous to use electrocardiogram (ECG) gating to identify the diastolic phase, and to select diastolic frames of the Doppler color flow IVUS image for detailed quantitative analysis.

In the peripheral arteries, however, where flow occurs predominantly during systole and where tissue motion is less significant, systolic gated frames may be more appropriate for detailed quantitative analysis.

In color flow imaging applications throughout most parts of the body (e.g., but not by way of limitation, hepatic, carotid, or peripheral artery), the tissue motion is negligible, so the velocity threshold for classification of an echo as a moving blood echo can be very low. In the case of coronary imaging, however, the tissue motion can be quite prominent because the coronary vessels overlie the heart muscle, thereby making it more difficult to distinguish tissue motion from blood flow. Although the motion of the heart muscle is quite rapid during early systole when the ventricles rapidly contract, the IVUS catheter 100 tends to move with the heart by virtue of its position within the coronary artery. Thus, the relative motion between the catheter and the surrounding tissue is usually significantly less than the absolute motion of the heart.

An example of a fast movement of the IVUS catheter 100 with respect to the heart would be for the catheter to shift one vessel diameter (approximately 3 millimeters) during the approximately 100 milliseconds that constitutes the early portion of systole. The corresponding relative tissue velocity in this case would be approximately 3 centimeters per second. Throughout most of the cardiac cycle, and in the majority of locations throughout the epicardial arterial tree, the actual tissue velocity will be much less than this maximum likely tissue velocity estimate. Thus, for this additional reason, it may be beneficial to obtain a diastolic gated flow measurement to more accurately obtain blood flow velocities.

In addition, the principles of the above described imaging system and methods can be applied to imaging systems based on other types of waves, such as electromagnetic waves (light, or radio waves), wherein the waves might be emitted/received by an angled emitter/receiver or deflected by an angled mirror, such that the waves propagate at an angle substantially tilted away from a perpendicular to the axis of the catheter. More specifically, while the above disclosure discusses application of the concepts to emitters/receivers that continuously rotate 360° in a single direction, it will be understood that the same methods can be applied to oscillating emitters/receivers. In oscillating systems, a transducer or mirror may be controlled to oscillate between 90° to 400° with preferred ranges being approximately 120° to approximately 360°. Still further, while the description above is set forth in relation to use of an ultrasound transducer, other forms of wave emitters/receivers such lasers or light sources may be controlled to take advantage of the systems and methods described above.

The above described imaging system is disclosed in a non-limiting example of at least one application for use as an intravascular ultrasound system for imaging blood vessels. It will be understood that blood vessels are only one type of structure within a living body that may be imaged by the described system in accordance with the methods set forth above. More specifically, fluid filled or surrounded structures, both natural and man-made, within a living body that may be imaged can include for example, but without limitation, structures such as: organs including the liver, heart, kidneys, gall bladder, pancreas, lungs; ducts; intestines; nervous system structures including the brain, dural sac, spinal cord and peripheral nerves; the urinary tract; as well as valves within the blood or other systems of the body. In addition to imaging natural structures, the images may also include imaging man-made structures such as, but without limitation, heart valves, stents, shunts, filters and other devices positioned within the body.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An imaging system, at least partially insertable into a structure of a living body, the system comprising:
an elongate member having a longitudinal axis extending along a distal portion, the elongate member having an energy emitter and echo receiver mounted adjacent the distal portion at an angle relative to the longitudinal axis such that an energy pulse generated by the energy emitter propagates from the elongate member at a non-perpendicular angle relative to the longitudinal axis, the echo receiver configured to collect velocity and amplitude data, the elongate member including a plurality of conductors extending between the energy emitter and echo receiver disposed adjacent the distal portion and a connection assembly disposed adjacent an opposite proximal portion of the elongate member;
an actuator coupled to the energy emitter, the actuator configured to move the energy emitter through a series of positions extending over at least a portion of a revolution; and
a control system coupled to the connection assembly, the control system configured to control the position of the energy emitter and the sequence of energy pulses generated by the energy emitter, the control system receiving the velocity and amplitude data from the echo receiver through the plurality of conductors and processing the velocity and amplitude data to generate an image of the structure.

2. The system of claim 1, wherein the energy emitter is an ultrasound transducer operable at approximately 40 MHz mounted to a drive shaft at an angle between 80 and 60 degrees relative to the longitudinal axis of the elongate member.

3. The system of claim 1, further including a drive shaft extending between the actuator and the energy emitter, the drive shaft extending substantially the entire length of the elongated member, the actuator rotating the drive shaft and energy emitter about the longitudinal axis.

4. The system of claim 1, wherein the image of the structure represents the amplitude ultrasound data in a grey-scale image and the velocity ultrasound data in overlaid color.

5. The system of claim 1, further including an encoder associated with said actuator generating an encoder pulse and a sequencer, wherein the sequencer is configured to generate a sequence of uniformly spaced transmit energy pulses per each encoder pulse and thereby generate a sequence of return echoes collected by the echo receiver.

6. The system of claim 5, further including an echo processor configured to process the sequence of return echoes to generate a single composite amplitude ray and to calculate the Doppler-derived velocity corresponding to each position along the ray by comparing the return echoes within the sequence.

7. The system of claim 6, further including signal processing hardware configured to extract the phase from the velocity data and generate a velocity estimate for reflectors at each pixel of the ray based on a rate of phase change between successive return echoes in the sequence.

8. The system of claim 7, wherein the control system utilizes the velocity estimate to form a hybrid structure image by overlaying a mask that colorizes portions of a grey-scale image representing amplitude where the velocity estimate is above a threshold value.

9. The system of claim 8, wherein the threshold value is approximately 3 centimeters per second.

10. The system of claim 5, wherein the encoder has 512 equally spaced radial positions and each sequence includes at least four energy pulses.

11. The system of claim 10, wherein each sequence includes up to sixteen energy pulses.

12. The system of claim 1, wherein the energy emitter is an ultrasound transducer and a first ultrasound pulse echo return within a sequence is acquired using a low gain setting and the remaining ultrasound pulse echo returns in the sequence are acquired using a high gain setting.

13. The system of claim 12, wherein the first ultrasound pulse echo return is processed separately to form a low gain amplitude ray and the remaining returns are processed together to form a composite amplitude ray, the low gain amplitude ray and the composite amplitude ray being combined to form a wide dynamic range ray, and a plurality of such wide dynamic range rays together forming a wide dynamic range structure image.

14. The system of claim 1, wherein the actuator oscillates the energy emitter along the portion of a revolution.

15. A rotational ultrasound catheter, the catheter comprising:
an elongate imaging core having a longitudinal axis and configured to rotate about the longitudinal axis;
an ultrasound transducer mounted to the imaging core at a non-orthogonal angle relative to the longitudinal axis of the imaging core such that an ultrasound beam emerges from the transducer at an angle between 10 and 30 degrees relative to a perpendicular to the longitudinal axis, the transducer configured to rotate in unison with the imaging core.

16. A method of imaging a structure within a living body, the method comprising:
positioning an elongate member having a distal portion with a longitudinal axis into the living body adjacent the structure to be imaged, the catheter including an ultrasound transducer movably mounted within the distal portion;
emitting a sequence of ultrasound pulses from the transducer at a substantially non-perpendicular angle relative to the longitudinal axis while moving the transducer through at least a portion of a revolution with respect to the longitudinal axis;
receiving a sequence of ultrasound return echoes from structure features including fluid within the structure;
processing the sequence of ultrasound echoes to generate a single composite amplitude ray associated with a position along the portion of the revolution;
processing the sequence of ultrasound echoes to determine the velocity of structure features;
and displaying a structure image representing velocity and amplitude information.

17. The method of claim 16, wherein the vessel image includes a grey-scale representation of amplitude information and a color representation of velocity information.

18. The method of claim 16, wherein the structure image includes a grey-scale representation of amplitude information and brightness is diminished for pixels in the grey-scale image for pixels where the velocity estimate for the pixel is above a threshold velocity.

19. The method of claim 18, wherein the threshold level is approximately 3 centimeters per second.

20. The method of claim 16, wherein determining the velocity is based on a rate of phase change between successive ultrasound echoes within a sequence.

21. The method of claim 16, wherein the portion of a revolution is divided into a number of equally spaced segments each designated by an encoder pulse, and said emitting occurs upon a receipt of an encoder pulse.

22. The method of claim 21, wherein each composite amplitude ray is associated with an encoder pulse.

23. The method of claim 16, wherein moving the transducer includes rotating the transducer about the longitudinal axis in a continuous motion through 360 degrees.

24. A method of quantitatively assessing the fluid flow of a structure within a living body, the method comprising:
positioning an elongate catheter having a longitudinal axis within the lumen of a structure, the catheter including an ultrasound transducer movably mounted within the catheter;
emitting ultrasound beams and receiving ultrasound echoes at a substantially nonperpendicular angle relative to the longitudinal axis;
constructing a grey-scale IVUS image of the structure based on the ultrasound echoes;
calculating the velocity estimates for a plurality of pixels forming the grey-scale image; and
determining quantitative fluid flow within the structure by using the velocity estimates in combination with physical anatomic measurements of the structure from the grey-scale image.

25. The method of claim 24, wherein calculating the velocity estimates for the pixels of the grey-scale image is based on a rate of phase change between successive ultrasound echoes.
